(19) United States
(12) Patent Application Publication
(10) Pub. No.: US 2020/0029993 A1
(43) Pub. Date: Jan. 30, 2020

(54) TISSUE EXCISION INSTRUMENT

(71) Applicant: Shoichi NAKAMURA, Nagano-ken (JP)

(72) Inventor: Shoichi NAKAMURA, Nagano-ken (JP)

(73) Assignees: Shoichi NAKAMURA, Nagano-ken (JP); ACP JAPAN CO., LTD., Tokyo (JP)

(21) Appl. No.: 16/046,388

(22) Filed: Jul. 26, 2018

(51) Int. Cl. A61B 17/3205; A61B 10/02

(52) U.S. Cl. CPC A61B 17/3205; A61B 10/0266; A61B 17/32053

(57) ABSTRACT

The present invention is to provide a tissue excision instrument used for medical purposes such as diagnosis, curing, or examination of a living body and excellent in operability. A cylindrical blade part has an annular-shaped blade edge externally sharpened such that the diameter thereof is reduced more toward the tip relative to a diameter d of a blade part body. The blade edge has three curved parts provided at equal intervals along the circumference. The curved parts are irregularities which are formed along the axial direction of the blade part by arc-shaped gentle curves, by which a plurality of arches are formed on the circumference of the blade edge.

### BACKGROUND OF THE INVENTION

### Field of the Invention

The present invention relates to a tissue excision instrument for excising a part of body tissue including skin.

### Description of the Related Art

In medical sites, tissue excision instruments for excising or sampling a part of body tissue (hereinafter, referred to merely as “tissue” in this specification) have been used for the purpose of diagnosing, curing, or examining the tissue including skin of a living body.

For example, such a tissue excision instrument has a cylindrical blade edge, and a user excises a part of tissue with the blade edge (see, for example, Patent Document 1).

As illustrated in FIG. 9, such a tissue excision instrument 100 as a conventional type is constituted of a cylindrical handle part 101 and a blade part 102. An annular blade edge of the blade part 102 is pressed perpendicularly against a tissue surface 103, and then the blade part 102 is rotated, whereby the tissue is excised in a circular shape.

However, when the annular blade edge is pressed against the tissue, pressing force caused by the pressing operation of the handle part 101 is dispersed circularly, so that the force of appropriate magnitude is required for resisting elasticity of the tissue, resulting in difficulty in operation.

In order to cope with this, a tissue excision instrument is proposed in which the tip flat surface of the blade edge of the blade part 102 perpendicular to the axial direction of the handle part 11 is inclined as illustrated in FIG. 10A or made corrugated as illustrated in FIG. 10B so as to allow first the pressing force to concentrate on a part of the tissue to be excised circularly (see, for example, Patent Document 2).
[Patent Document 1] Japanese Patent Application Publication No. 2000-126196
[Patent Document 2] Japanese Patent Application Publication No. 2006-149860

However, in the case of the excision instrument illustrated in FIG. 10A, when forming a cut in the tissue, the length of the cut is increased by the inclination of the blade edge. This may cause the tissue to be excised unnecessarily deeply, thus requiring fine control of force.

Further, in the case of the excision instrument illustrated in FIG. 10B, it is difficult to excise the tissue in a fine circular shape due to resistance from the tissue when the tip of the corrugated blade edge is pierced into the tissue and then rotated, which may make the cutting plane of the tissue uneven.

### SUMMARY OF THE INVENTION

The present invention has been made in view of the problems of the conventional techniques, and the object thereof is to provide a tissue excision instrument having an improved cutting quality of the annular blade edge.

To solve the above problems, according to the present invention, there is provided a tissue excision instrument in which the tip of a cylindrical blade part is pressed against tissue to excise the tissue extracted and led into the cylinder of the blade part, wherein a plurality of curved parts having arc-shaped irregularities are uniformly formed on the circumference of an annular-shaped blade edge of the blade part. In this case, the curvatures of the arc-shapes of the respective curved parts are preferably the same.

By providing a pushing part that is axially moved inside a hollow of the blade part to protrude outside from the blade part, it is possible to easily remove a tissue slice adhering to the inside of the blade edge after excision.

Further, in a tissue excision instrument having a punching part configured to protrude from and retract into the inside of the blade edge, it is preferable to provide, around the end face of the punching part that faces the blade edge, a second blade part that contacts the inside of the blade edge when the punching part is moved down.

In the tissue excision instrument according to the present invention, when the blade part pressed against the tissue is rotated, a plurality of arch-shaped irregularities provided on the annular-shaped blade edge of the blade part act as arches, so that the tissue is finely excised, and operability is enhanced.

### BRIEF DESCRIPTION OF THE DRAWINGS

FIG. 1 is a side view illustrating the entire configuration of a tissue excision instrument according to a first embodiment of the present invention;

FIG. 2A is a cross-sectional side view of a blade part of the tissue excision instrument illustrated in FIG. 1, and FIG. 2B is a plan view thereof;

FIGS. 3A and 3B are views each illustrating the outer appearance of a tissue excision instrument according to a second embodiment of the present invention;

FIG. 4 is a view illustrating the outer appearance of a pushing member of the tissue excision instrument illustrated in FIGS. 3A and 3B;

FIG. 5A is a cross-sectional side view of a blade part of the tissue excision instrument illustrated in FIGS. 3A and 3B, and FIG. 5B is a plan view thereof;

FIG. 6 is a side view illustrating the entire configuration of a tissue excision instrument according to a third embodiment of the present invention;

FIGS. 7A to 7C are main part side views each illustrating a tissue excising operation by the tissue excision instrument according to the third embodiment of the present invention;

FIG. 8 is a side view illustrating a main part of a tissue excision instrument according to a modification of the third embodiment;

FIG. 9 is a view illustrating the configuration of a conventional general tissue excision instrument; and

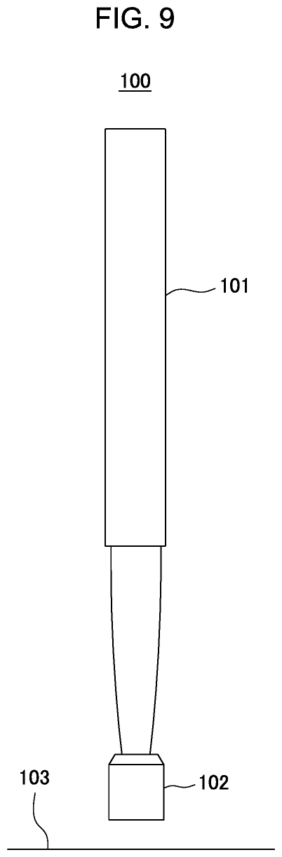

FIGS. 10A and 10B are cross-sectional side views each illustrating an example of the shape of a blade edge of a blade part in the conventional tissue excision instrument.

### DESCRIPTION OF THE PREFERRED EMBODIMENTS

Hereinafter, embodiments of the present invention will be described with reference to the drawings.

### First Embodiment

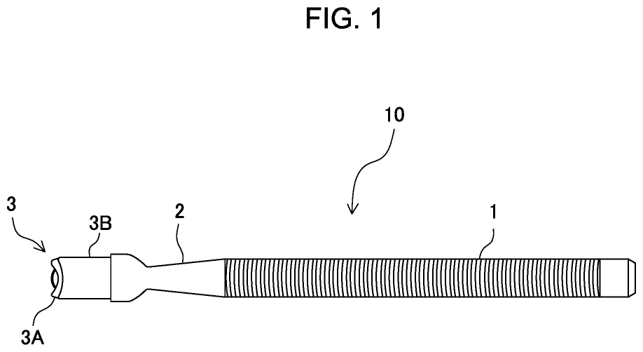

FIG. 1 is a side view illustrating the entire configuration of a tissue excision instrument according to the present invention. As illustrated in FIG. 1, a tissue excision instrument 10 according to the present embodiment has a handle part 1, a blade holder 2 provided at the end portion of the handle part 1, and a blade part 3 positioned at the tip of the blade holder 2. The handle part 1, blade holder 2, and blade part 3 are fixed so as to be aligned substantially linearly.

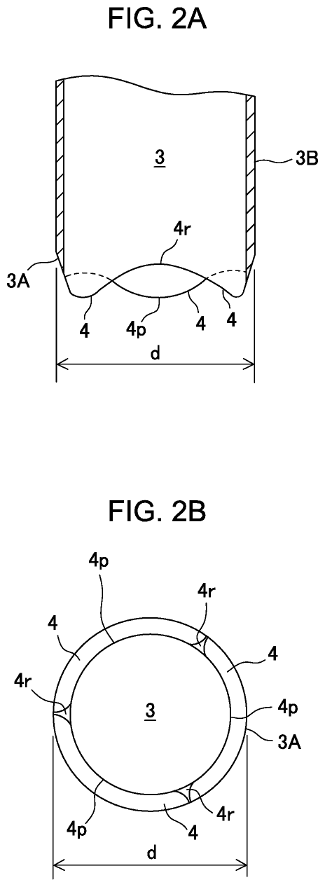

FIG. 2A is a cross-sectional side view of the blade part 3, and FIG. 2B is a plan view of the blade part 3. The blade part 3 is formed into a cylindrical shape, and a blade edge 3A is externally sharpened such that the diameter of the outer peripheral surface thereof is reduced more toward the tip relative to a diameter d of a blade part body 3B.

The annular-shaped blade edge 3A has three curved parts 4 provided at equal intervals along the circumference. The curved parts 4 are irregularities which are formed along the axial direction of the blade part 3 by arc-shaped gentle curves, by which a plurality of arches are formed on the circumference of the blade edge 3A. In this case, the arches, i.e., the arc-shaped curved parts 4 preferably have the same curvature.

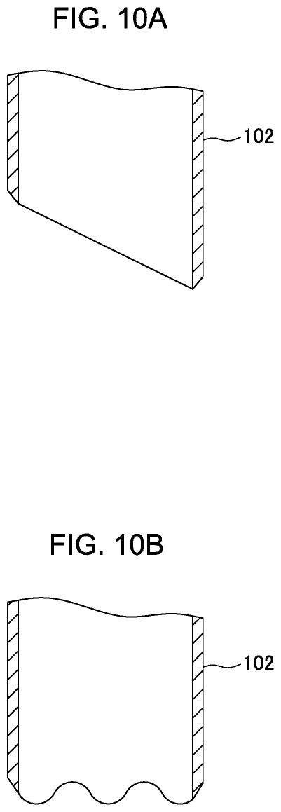

By providing the curved parts 4 that form the arches on the blade edge 3A, it is possible to form a smooth cut in the tissue by the arch which is a gentle curved portion of the curved part 4 extending from a peak 4p to a valley 4r of the curve when the blade edge 3A is pressed perpendicularly against a tissue surface. In order to form such an arch on the circumference of the blade edge 3A, it is necessary to determine the maximum number of the curved parts 4 to be provided on the blade edge 3A in accordance with the diameter of the annular ring of the blade edge 3A. In this example, the diameter of the annular ring of the blade edge 3A is 6 mm. In this case, up to four curved parts 4 can be provided. However, when the number of the curved parts 4 is equal to or larger than 4, the arch cannot be formed on the circumference of the blade edge 3A, that is, the blade edge has a corrugated shape as illustrated in FIG. 10B, thus making it impossible to form a smooth cut in the tissue.

The handle part 1 is a part that a user holds when using the tissue excision instrument 10 and is a straight rod-like member made of metal or resin. The handle part 1 is subjected to knurling or satin treatment so as to prevent slipping of a hand of a user when he or she holds the handle part 1.

The blade holder 2 is a member which is integrated with the blade part 3. Specifically, at the time of manufacturing, the blade part 3 is loaded into a die, and then molten resin is injected to wrap the blade part 3, followed by solidification of the molten resin. In this case, the handle part 1 may also be made of the same resin and molded integrally with the blade holder 2 in the die.

In the thus configured tissue excision instrument 10, the cylindrical blade part 3 is pressed against the tissue, and the handle part 1 is rotated. With the rotation of the handle part 1, the blade part 3 is rotated. Then, in the blade edge 3A, by the arch formed by a part of each of the curved parts 4 extending from the peak 4p to the valley 4r, a cut having a uniform depth can be formed in the tissue along the circle of the blade edge 3A. At this time, by the plurality of arches provided at equal intervals, the rotation of the handle part 1 is transmitted smoothly to the blade part 3, enhancing operability.

A tissue slice extracted and led into the cylinder of the blade part 3 and excised there adheres to the inside of the blade edge 3A. Then, the tissue excision instrument 10 is separated from the tissue. In this manner, the tissue slice can be excised from the tissue and sampled.

The thus configured tissue excision instrument 10 is used for an operation such as removal of a mole or sampling of a part of tissue as an inspection sample.

### Second Embodiment

In the tissue removal process using the above tissue excision instrument 10, there may be a case where it is necessary to insert tweezers or the like into the blade part 3 in order to remove the excised tissue slice adhering to the inside of the blade edge 3A. Such a removal operation takes labor and, in particular, when the diameter of the annular ring of the blade edge 3A is small, the removal operation is very troublesome.

The following describes an embodiment of the tissue excision instrument capable of quickly removing the tissue slice adhering to the inside of the blade edge.

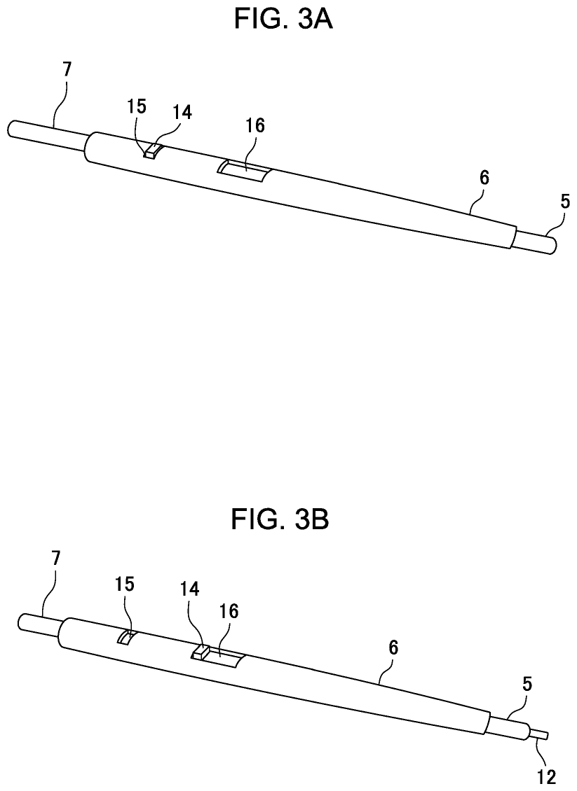

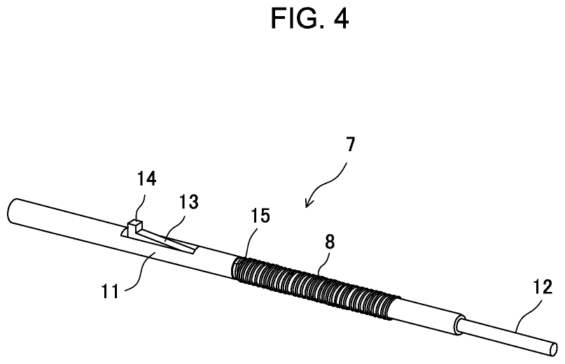

A tissue excision instrument 20 illustrated in FIGS. 3A and 3B is constituted of a cylindrical cover part 6 provided with a blade part 5 at the tip thereof, a pushing member 7 inserted into the cover part 6 at the time of assembly and configured to be axially slidably movable, and a spring member 8 (see FIG. 4) engaged between the cover part 6 and the pushing member 7.

The pushing member 7 is a resin integral molding and constituted of a rod-like shaft body 11, a pushing part 12, and a lock part 13 as illustrated in FIG. 4. The lock part 13 branching from the shaft body 11 in the longitudinal direction retains the end portion thereof isolated from the shaft body 11 at a position separated from the shaft body 11 by its own elastic force. The lock part 13 is provided with a protrusion 14 at the free-side end portion thereof. Further, a step 15 for locking the coil spring member 8 wound around part of the pushing member 7 is formed on the shaft body 11.

The cover part 6 has two lock holes 15 and 16 one of which is engaged with the protrusion 14 of the lock part 13 when the pushing member 7 is inserted into the cylinder.

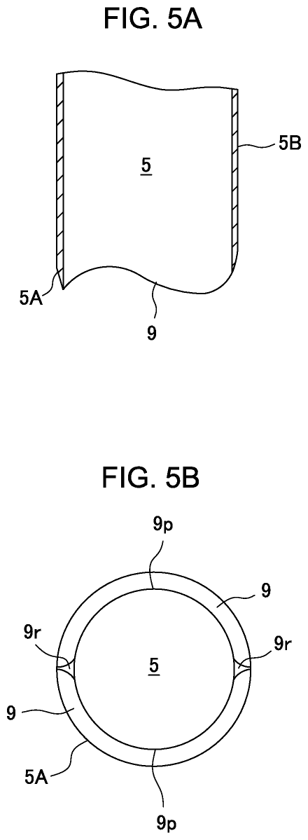

The blade part 5 is formed integrally with the cover part 6. Specifically, at the time of molding, the blade part 5 is loaded into a die, and then molten resin is injected to wrap the blade part 5, followed by solidification of the molten resin. FIG. 5A is a cross-sectional side view of the blade part 5, and FIG. 5B is a plan view of the blade part 5. The blade part 5 is formed into a cylindrical shape, and a blade edge 5A is externally sharpened such that the diameter thereof is reduced more toward the tip relative to the diameter of a blade part body 5B.

The annular-shaped blade edge 5A has a plurality of curved parts 9 provided at equal intervals along the circumference. The diameter of the annular ring at the tip of the blade edge 5A in this example is set to 3 mm. When the diameter of the annular ring is set to such a small value, the maximum number of the curved parts 9 each having an arc-shaped gentle curve is set to about three (FIGS. 5A and 5B illustrate a case where two curved parts 9 are formed).

Normally, as illustrated in FIG. 3A, in the thus configured tissue excision instrument 20, the protrusion 14 is locked into the lock hole 15, and thus the pushing part 12 at the tip of the pushing member 7 does not advance inside the cylindrical blade part 5. In this state, the cover part 6 is rotated with the blade part 5 pressed against the tissue to rotate the blade part 5. Then, in the blade edge 5A, the tissue is circularly cut by the arch formed by a part of each of the curved parts 9 extending from a peak 9p to valley 9r, with the result that an extracted and excised tissue slice in the cylinder of the blade part 3 adheres to the inside of the blade edge 5A.

To collect the tissue slice adhering to the inside of the blade edge 5A, the protrusion 14 is pushed and, at the same time, the rear end of the pushing member 7 is pushed in such a way that the pushing member 7 is moved toward the blade edge 5A against the spring force of the spring member 8. As a result, the locking state between the protrusion 14 of the lock part 13 and the lock hole 15, which is held by the elastic force of the lock part 13 itself, is released, and the lock part 13 moves inside the cover part 6. Then, when the protrusion 14 reaches the lock hole 16, the lock part 13 is returned in a direction separated from the shaft body 11 by its own elastic force, and the protrusion 14 is locked into the lock hole 16.

When the pushing member 7 is moved until the protrusion 14 is locked into the lock hole 16, the pushing part 12 protrudes outward from the tip of the blade part 5 as illustrated in FIG. 3B, with the result that the tissue slice adhering to the inside of the blade edge 5A is pushed outside and collected. When the protrusion 14 locked into the lock hole 16 is pushed toward the inside of the cover part 6, the locking state between the protrusion 14 and lock hole 16 is released. Then, the pushing member 7 is moved until the protrusion 14 is locked once again into the lock hole 15 by the restoration force of the spring member 8, and the pushing part 12 retracts from the inside of the blade part 5.

### Third Embodiment

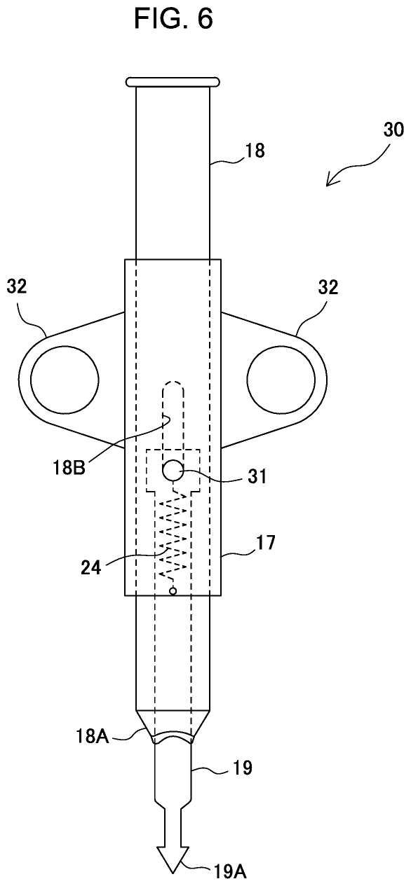

The following describes an embodiment of a puncher 30 which is a tissue excision instrument used for punching a hole in a blood vessel during medical operation. FIG. 6 illustrates the configuration of the puncher 30. The puncher 30 includes a cylindrical outer case 17, a cylindrical blade part 18 retained inside the outer case 17, and a punching part 19 retained inside the blade part 18.

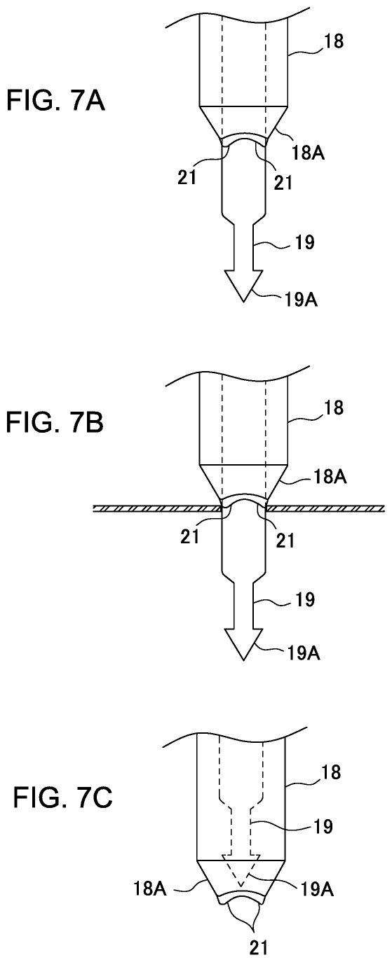

The outer case 17 has, on both side faces thereof, a finger hook 32 having a hole through which a finger of an operator is inserted. The blade part 18 has an annular-shaped blade edge 18A at the tip thereof, and the blade edge 18A has three curved parts 21 provided at equal intervals along the circumference. As illustrated in FIG. 7, the curved parts 21 are irregularities which are formed along the axial direction of the blade part 18 by arc-shaped gentle curves, by which a plurality of arches are formed on the circumference of the blade edge 18A.

The punching part 19 having a columnar body has a circular-cone-shaped pointed head part 19A at the tip thereof. The end portion of the punching part 19 on the side opposite to the pointed head part 19A is fixedly supported to the outer case 17 by a pin 31. The blade part 18, together with the punching part 19, is also supported by the outer case 17 by means of the pin 31.

The blade part 18 has an elongated hole 18B extending in the longitudinal direction thereof, and the pin 31 is slidably fitted into the elongated hole 18B. The pin 31 is biased by a spring 24 and is normally locked to the lower portion of the elongated hole 18B. In this state, as illustrated in FIG. 7A, the lower end portion of the punching part 19 including the pointed head part 19A protrudes outward from the blade edge 18A.

In this state, an operator presses the entire puncher 30 against a blood vessel to cause the tip of the punching part 19 to rush into the blood vessel while breaking through the blood vessel surface with the pointed head part 19A. Then, when the operator manually pushes down the upper end of the blade part 18 against the biasing force of the spring 24 while holding the outer case 17 at the finger hook 32, the blade part 18 is moved until the pin 31 is locked to the upper portion of the elongated hole 18B and, as illustrated in FIG. 7C, the pointed head part 19A is housed inside the annular ring of the blade edge 18A. Thus, in a process that the pointed head part 19A is housed inside the blade edge 18A, the tissue of the blood vessel surface into which the pointed head part 19A is pierced is excised to be punched.

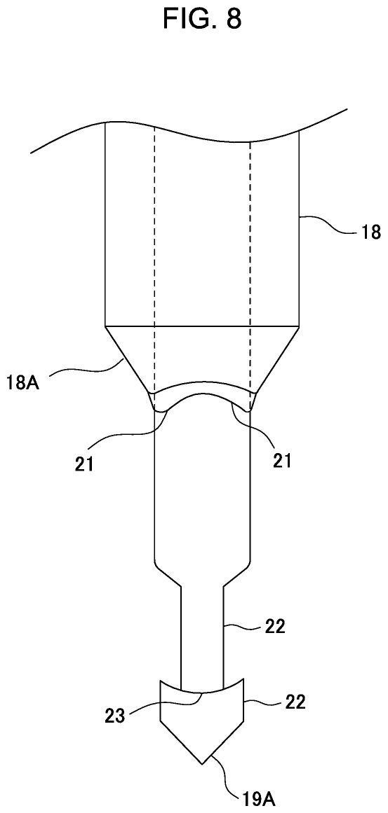

FIG. 8 illustrates a modification of the puncher 30 of FIG. 6. The pointed head part 19A has a second blade part 22 having a curved part 23 at its upper end potion that contacts the inside of the annular-shaped blade edge 18A when the blade part 18 is moved down. In the thus configured puncher 30, when the pointed head part 19A is housed inside the annular ring of the blade edge 18A, the blade part 22 of the pointed head part 19A is slid inside the blade edge 18A to finely excise the tissue of the blood vessel surface into which the pointed head part 19A is pierced, whereby the blood vessel surface can be hollowed out. While, in FIG. 8, the blade edge 18A is externally sharpened and the blade part 22 is internally sharpened, they may be designed in the opposite way.

While the embodiments of the present invention have been described, the present invention is not limited to the above embodiments. Various modifications can be made based on the technical idea of the present invention, and the present invention does not exclude such modifications. For example, the curvatures of the arc-shapes of the respective curved parts 4 need not be the same as long as the arches are ensured. Further, the blade edge may be internally sharpened such that the diameter of the inner peripheral surface thereof is reduced more toward the tip relative to the diameter of a blade part body.

The present invention relates to a tissue excision instrument used for medical purposes, such as diagnosis, curing, or examination of a living body and has industrial applicability.

1. A tissue excision instrument in which the tip of a cylindrical blade part is pressed against tissue to excise the tissue extracted and led into the cylinder of the blade part, wherein
a plurality of curved parts having arc-shaped irregularities are uniformly formed on the circumference of an annular-shaped blade edge of the blade part.

2. The tissue excision instrument according to claim 1, wherein
the curvatures of the arc-shapes of the respective curved parts are the same.

3. The tissue excision instrument according to claim 1, comprising a pushing part that is axially moved inside a hollow of the blade part to protrude outside from the blade part.

4. The tissue excision instrument according to claim 1, further comprising a punching part configured to protrude from and retract into the inside of the blade edge, wherein
a second blade part that contacts the inside of the blade edge when the punching part is moved down is provided around the end face of the punching part that faces the blade edge.
